(19) United States
(12) Patent Application Publication
(10) Pub. No.: US 2018/0066247 A1
(43) Pub. Date: Mar. 8, 2018

(54) BIOLOGICAL TISSUE GRINDING CONTAINER

(71) Applicant: Toshiba Medical Systems Corporation, Otawara-shi (JP)

(72) Inventors: Hiroaki Kushima, Otawara (JP); Nobuo Kawamura, Nasushiobara (JP); Tetsuya Kuwabara, Nasushiobara (JP)

(73) Assignee: Toshiba Medical Systems Corporation, Otawara-shi (JP)

(21) Appl. No.: 15/696,477

(22) Filed: Sep. 6, 2017

(30) Foreign Application Priority Data
Sep. 6, 2016 (JP) 2016-173670

(51) Int. Cl. C12N 15/10; B02C 19/16

(52) U.S. Cl. CPC C12N 15/1003; B02C 19/16

(57) ABSTRACT

According to one embodiment, a biological tissue grinding container includes a container portion and a vibrated portion, and vibration is transmitted to biological tissue from the vibration portion to grind the biological tissue. The vibrated portion includes a contact portion to be brought into direct contact with the biological tissue and defines a chamber which stores the biological tissue to be ground, together with the container portion. The vibrated portion is fixedly supported to be vibratable to the container portion directly or via a support portion provided between the container portion and itself, and has solidity higher than that of the container portion or support portion.

### CROSS-REFERENCE TO RELATED APPLICATIONS

This application is based upon and claims the benefit of priority from Japanese Patent Application No. 2016-173670, filed Sep. 6, 2016, the entire contents of which are incorporated herein by reference.

### FIELD

Embodiments described herein relate generally to a biological tissue grinding container for grinding biological tissue of animals, plants or microorganisms, bacteria, fungi, or virus to extract nucleic acid (DNA and/or RNA) from inside.

### BACKGROUND

There various types of procedures for physically or chemically grinding or crushing biological tissue of animals, plants or microorganisms, bacteria, fungi or virus (to be generally referred to as biological tissue hereafter) to extract nucleic acid. Chemical procedures, generally, entail drawbacks of requiring the cleaning process as post treatment, by which it usually takes time to extract nucleic acid and also raising the cost for the treatment. On the other hand, physical procedures are usually considered to be low-cost and efficient in grinding biological tissue to extract nucleic acid as long as the heat accompanying the grinding affects a cell or a virus not to denature its composition.

As a general physical grinding technique, a method of applying directly ultrasonic waves to an object of extraction is known, a typical example of which is the homogenizer. Further, the method of dipping a container which stores biological tissue in water and applying supersonic vibration thereto has been practically used.

The method of applying ultrasonic waves directly to an object for extraction entails a drawback that biological tissue may scatter to contaminate the surrounding environment, and therefore it requires measures to avoid the contamination of the peripheral environment. Moreover, the method of dipping a container which stores biological tissue entails a drawback of damping of vibration and accordingly, making the vibration uneven, which results in uneven grinding effect.

Thus, there is a demand for development of biological tissue grinding containers which can efficiently grind the biological tissue while avoiding contamination of surrounding environment by applying vibration to a biological tissue in a closed space and suppressing the damping to a minimum level.

### BRIEF DESCRIPTION OF THE DRAWINGS

FIG. 1A is a top view schematically showing a biological tissue grinding container to be mounted on a grinding device, and a vibration generator in a structural arrangement according to an embodiment.

FIG. 1B is a front view schematically showing the biological tissue grinding container to be mounted on the grinding device, and the vibration generator in the structural arrangement according to the embodiment.

FIG. 2 is a front view schematically showing a biological tissue grinding container to be mounted on a grinding device, and a vibration generator in a structural arrangement according to another embodiment.

FIG. 3 is a front view schematically showing a biological tissue grinding container to be mounted on a grinding device, and a vibration generator in a structural arrangement according to still another embodiment.

FIG. 4 is a front view schematically showing a biological tissue grinding container to be mounted on a grinding device, and a vibration generator in a structural arrangement according to still another embodiment.

FIG. 5 is a front view schematically showing a structure of a biological tissue grinding container to be mounted on a grinding device according to still another embodiment.

FIG. 6 is a front view schematically showing a structure of a biological tissue grinding container to be mounted on a grinding device according to still another embodiment.

FIG. 7 is an explanatory diagram illustrating the relationship between a vibration frequency of a vibration chip and a resonance frequency of a vibration chip junction in a biological tissue grinding container according to an embodiment.

### DETAILED DESCRIPTION

Various embodiments will be described hereinafter with reference to the accompanying drawings.

In general, according to one embodiment, there is provided a biological tissue grinding container comprising a container portion comprising a chamber which stores biological tissue to be ground; and a vibrated portion comprising a contact portion to be brought into direct contact with the biological tissue and defining the chamber together with the container, which extend to outside of the container portion, the vibrated portion transmitting vibration to the biological tissue to grind the biological tissue.

### Embodiment 1

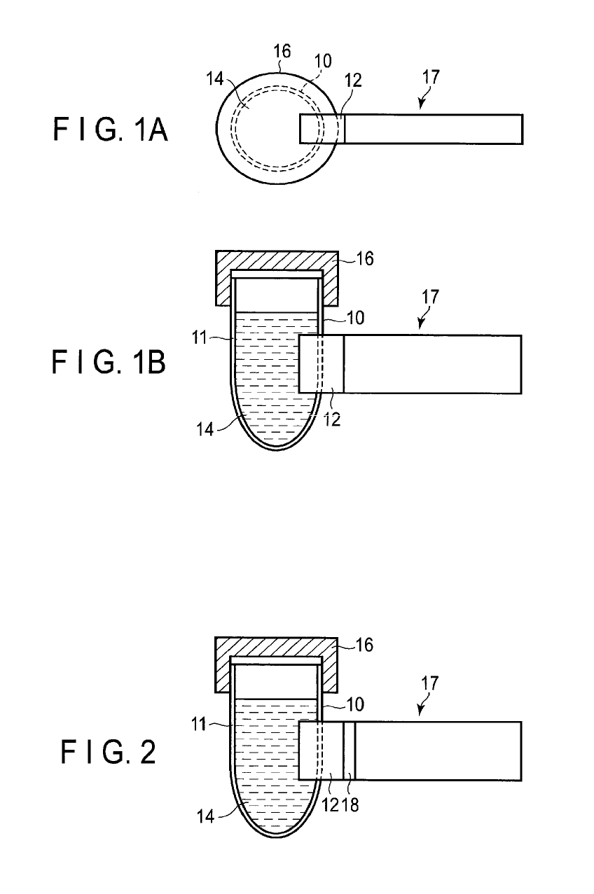

FIGS. 1A and 1B schematically show a structural arrangement of a biological tissue grinding container 10 mounted to a grinding device according to the embodiment, and a vibration generator 17 which applies vibration to the biological tissue grinding container 10.

The biological tissue grinding container 10 has, for example, a bottomed cylindrical shape as a whole, with an opening formed in an upper portion thereof and a container chamber in a middle portion. Into the biological tissue grinding container 10, a test object of biological tissue of an animal, plant or microorganism, bacteria, fungi or a virus (to be simply referred to as biological tissue hereinafter in the specification) is located together with a liquid through the opening. The liquid in which the biological tissue is contained is called a sample solution 14. When the biological tissue grinding container 10 is mounted to the grinding device, the opening of the biological tissue grinding container 10 is sealed in a lid 16 so as to prevent the sample solution 14 contained therein from dispersing outside by vibration applied from the outside.

The biological tissue grinding container 10 comprises a container portion 11 which store a biological tissue, and a tabular solid vibration chip 12 as a vibrated portion. The vibration chip 12 is fixed by insertion to the container portion 11 so as not to be removed therefrom if the container portion 11 is deformed by being vibrated. In other words, the vibration chip 12 penetrates the container portion 11 and extends from inside to the outside of the container portion 11, exposing both end surfaces to the inside and the outside of the container portion 11. An inner surface of the vibration chip 12 is brought into direct contact with the biological tissue as a contacting portion, and thus defines the container chamber which stores a biological tissue together with the container portion 11. The container portion 11 is formed from a transparent flexible material, for example, a resin, which is movable in a minute range by vibration, whereas the vibration chip 12 is formed from a metal or ceramics which has solidity sufficiently higher than that of the container portion 11. The inner surface of the vibration chip 12 is exposed to the inside of the container portion 11 so as to be brought into direct contact with an biological tissue, and the outer surface side is removably coupled with the vibration generator 17 provided in the device. Here, the material of the vibration chip 12 may only be one different from that of the container portion 11 and having solidity higher than that of the container portion 11. The vibration chip 12 should only adopt such a material or supporting structure which allows the vibration chip 12 to vibrate while maintaining the airtightness of the container portion 11. The vibration generator 17 is tightly fixed to the outer surface side of the vibration chip 12 when the biological tissue grinding container 10 is mounted to the device. The vibration generator 17 comprises an ultrasonic oscillator which generates supersonic vibration and an ultrasonic horn which amplify the supersonic vibration generated by the ultrasonic oscillator to an optimal amplitude, and the oscillating end of the ultrasonic horn is coupled with the outer surface side of the vibration chip 12.

While the biological tissue grinding container 10 is mounted to and sealed by the grinding device (not shown), it is vibrated by the vibration generator 17. The vibration is transmitted to the solid vibration chip 12 to which the vibration generator 17 is mounted, and is directly propagated to the biological tissue in the sample solution 14 from the vibration chip 12 to vibrate the biological tissue. As a result, the biological tissue in the sample solution 14 is effectively ground. After the tissue grinding treatment, the biological tissue grinding container 10 is removed from the vibration generator 17 to the outside of the grinding device, to be replaced by a biological tissue grinding container 10 storing a new biological tissue, thus executing the grinding treatment for each of the biological tissue grinding containers 10.

While grinding, the biological tissues are effectively crushed by cavitation created within the biological tissue grinding container 10. Further, since the biological tissue grinding container 10 is closed by the lid portion 16 when mounting it to the grinding device, such an event that contaminates the surrounding environment can be prevented. Since the vibration generator 17 is coupled with the highly solid vibration chip 12, while grinding, vibration is directly transmitted to the biological tissue in the biological tissue grinding container 10 without substantially damping. As a result, biological tissue can be efficiently ground. Moreover, by simply mounting the biological tissue grinding container 10 to the device, the vibration generator 17 can be coupled with the vibration chip 12. Therefore, the time required to mount a biological tissue grinding container 10 and replace it with another one after a treatment and so on, can be shortened, thereby making it possible to shorten the testing time.

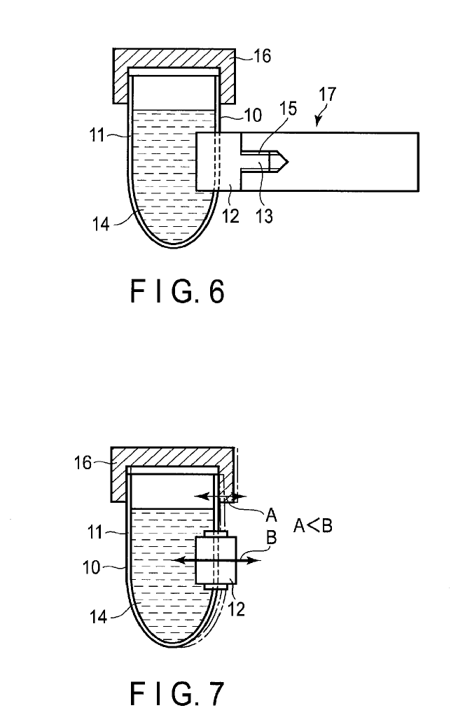

FIGS. 2 to 4 show various embodiments 2 to 4, respectively, of the coupling structure of the vibration generator 17 coupled with the biological tissue grinding container 10 shown in FIG. 1. FIGS. 5 and 6 show other embodiments 5 and 6 of the coupling structure of the biological tissue grinding container 10 and the vibration chip 12. FIG. 7 is a schematic diagram illustrating the biological tissue grinding container 10 shown in FIG. 1 and the vibration chip 12 while being vibrated. In the embodiments 2 to 7 described below, the same structural parts and sections as those of the embodiment 1 described above with reference to FIG. 1 will be designated by the same referential symbols, and detailed descriptions therefor will be omitted or abbreviated.

### Embodiment 2

In the grinding device shown in FIG. 2, a high-polymer resin film 18 is formed on an outer surface-side contact surface of a vibration chip 12, and a vibrating end of a vibration generator 17 is tightly attached to the contact surface.

According to the embodiment, the high-polymer resin film 18 is formed from an optimal material that does not block transmission of vibration or not wear out by the collision due to reciprocating movement of the vibrating end of the vibration generator 17. Therefore, high-frequency vibration can be efficiently transmitted to the vibration chip 12 without damaging the vibrating end of the vibration generator 17 to transmit vibration to the biological tissue in the biological tissue grinding container 10, thereby grinding the biological tissue.

### Embodiment 3

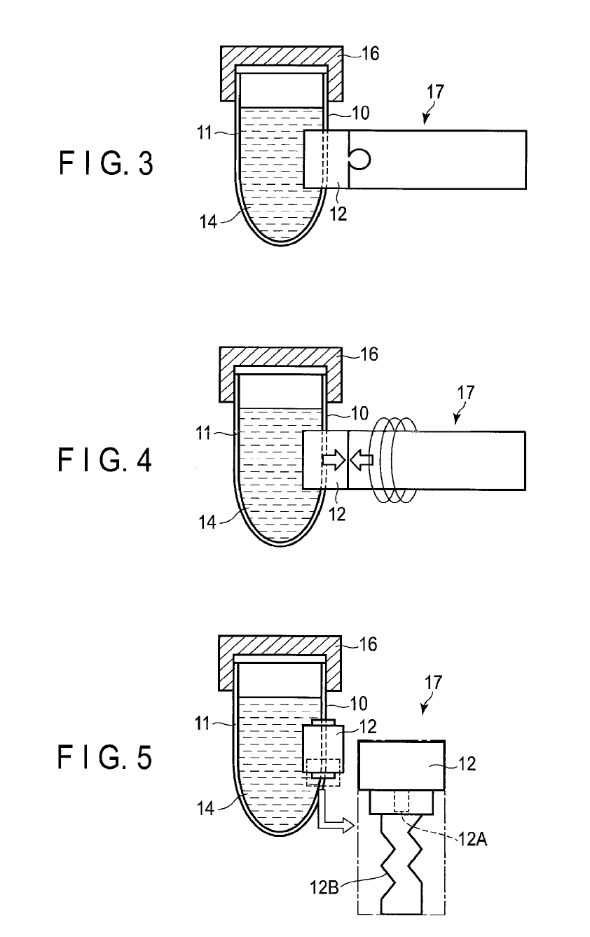

In a grinding device shown in FIG. 3, one of a projection or a recess, here, for example, the recess is firmed in a vibration generator 17, and the other one, for example, the projection is formed in an outer surface side of a vibration chip 12. The projection is fit into the recess to fixedly engage the vibration chip 12 and the vibration generator 17 with each other so as to vibrate integrally as one body. By adopting such structure, supersonic vibration can be efficiently transmitted to the vibration chip 12 without damaging the vibrating end of the vibration generator 17 to transmit vibration to the biological tissue in the biological tissue grinding container 10, thereby grinding the biological tissue.

### Embodiment 4

In a grinding device shown in FIG. 4, a vibration generator 17 and a vibration chip 12 are fixed together by attraction due to electromagnetic force. Here, the vibration chip 12 is formed from a magnetic material. The vibration generator 17 comprises an ultrasonic horn around which a wire is wound to impart the function of an electromagnet, and the electromagnet is excited externally, thus electromagnetically fixing the vibration generator 17 tightly to the vibration chip 12. With such structure, the vibration generator 17 and the vibration chip 12 can be reliably coupled with each other to be able efficiently transmission of ultrasonic waves, and therefore the biological tissue in the biological tissue grinding container 10 can be ground by supersonic vibration.

### Embodiment 5

In the biological tissue grinding container 10 shown in FIG. 1, the vibration chip 12 to the container portion 11 can be formed as one body, for example, by a technique of molding different types of materials together. More specifically, as shown in FIG. 5, a rib 12A projecting around an outer circumference of the vibration chip 12 is provided and the rib 12A is integrally molded to the container portion 11. Here, the rib 12A is fixed by being embedded in the container portion 11, and thus both are reliably fixed. Here, preferably, an adjacent joint portion of the container portion 11 should be provided adjacent to a joint portion between the vibration chip 12 and the container portion 11 so as for the container portion 11 to hold the vibration chip 12 reliably when the vibration chip 12 vibrates. The adjacent joint portion is formed on a bellows-type flex portion 12B to enable the adjacent joint portion of the container portion 11 to finely move with the vibration of the vibration chip 12. Thus, the biological tissue grinding container 10 comprises an inner surface portion including the inner surface side of the vibration chip 12, and the inner surface portion is vibrated by the vibration chip 12 to transmit the vibration directly to the biological tissue in the sample solution 14. As a result, ultrasonic waves can be efficiently transmitted to the inside of the container 11 from the vibration generator 17 to grind the biological tissue in the biological tissue grinding container 10 by supersonic vibration.

### Embodiment 6

As shown in FIG. 6, a male screw portion 13 may be formed in the vibration chip 12 and a female screw portion 15 in the vibration generator 17 so as for the female screw portion 15 to engage with the male screw portion 13 to mechanically couple these members with each other. The vibration generator 17 should be configured to have a screw mechanism (not shown) to be able to rotate around its central axis so as to engage the female screw portion 15 with the male screw portion 13. In place of the vibration chip 12 shown in FIG. 6, a female screw portion (not shown) may be formed in the vibration chip 12 and a male screw portion (not shown) may be formed in the vibration generator 17, and these screw portions may be engaged with each other to fix the vibration generator 17 and the vibration chip 12 together by coupling.

According to the structure where the vibration generator 17 and the vibration chip 12 are fixed by coupling with the screw mechanism, vibration of the vibration generator 17 can be reliably transmitted to the vibration chip 12 to transmit the vibration directly to the biological tissue in the sample solution 14. Since the vibration generator 17 and the vibration chip 12 are coupled together reliably, ultrasonic waves can be transmitted efficiently to grind the biological tissue in the biological tissue grinding container 10 by supersonic vibration.

### Embodiment 7

As shown in FIG. 7, in the grinding device, the material and the structure of the biological tissue grinding container 10 and the vibration chip 12 should preferably be determined so as to set an intrinsic vibration frequency A at the joint portion between the biological tissue grinding container 10 and the vibration chip 12, lower than a vibration frequency B applied to the vibration chip 12 by the vibration generator 17. When the intrinsic frequency A is set lower than the vibrational frequency B, ultrasonic waves can be efficiently transmitted to the inside of the sample solution 14, and the biological tissue in the biological tissue grinding container 10 can be ground by supersonic vibration.

According to the grinding devices of embodiments 1 to 7, vibration is applied to biological tissue within the closed biological tissue grinding container 10 while suppressing the damping to the minimum. Thus, it is possible to grind biological tissue efficiently while avoiding contamination of the surrounding environment.

In each of the grinding devices, the biological tissue grinding container 10 is mounted to the grinding device in a sealed manner, and vibration is transmitted from the vibration generator 17 to the solid vibration chip 12, and thus directly transmitted to biological tissue to be examined from this vibration chip 12, thereby vibrating the biological tissue. As a result, the biological tissue in the sample solution 14 is effectively ground. After the grinding treatment of biological tissue, the biological tissue grinding container 10 is removed from the vibration generator 17 to the outside of the grinding device, and replaced by another biological tissue grinding container 10 containing a new biological tissue. Thus, grinding treatment is executed for each biological tissue grinding container 10.

While grinding, a cavitation is created in the sample solution 14 in the biological tissue grinding container 10, and biological tissue is effectively ground by the cavitation. Further, when the biological tissue grinding container 10 is mounted to the grinding device, the biological tissue grinding container 10 is sealed, thereby avoiding such an even that contaminates the surrounding environment. Since the solid vibration chip 12 is coupled with the vibration generator 17, vibration is transmitted to the biological tissue in the biological tissue grinding container 10 without damping while grinding. As a result, biological tissue, bacteria, fungi or virus can be efficiently ground. Further, the vibration generator 17 can be coupled with the vibration chip 12 by simply mounting the biological tissue grinding container 10 to the device, and therefore the time required to mount a biological tissue grinding container 10 and detachment of the biological tissue grinding container 10 finished with grinding treatment can be shortened.

The solution in the biological tissue grinding container 10 in which the biological tissue is ground with the grinding device is taken out with a pipet or the like and then subjected to a post treatment which separates nucleic acid (DNA and/or RNA), to extract nucleic acid from solution. After that, the nucleic acid is subjected to amplification treatment, and the nucleic acid is specified.

Note that in various embodiments described above, when the object to be ground is a hard biological tissue, beads may be contained in the container portion 11 together with the biological tissue to grind the tissue by being brought into contact with the beads being vibrated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A biological tissue grinding container comprising:
a container portion comprising a chamber which stores biological tissue to be ground; and
a vibrated portion comprising a contact portion to be brought into direct contact with the biological tissue and defining the chamber together with the container, which extend to outside of the container portion,
the vibrated portion transmitting vibration to the biological tissue to grind the biological tissue.

2. The container of claim 1, wherein
the vibrated portion is fixedly supported to the container portion directly, and has a solidity higher than that of the container portion.

3. The container of claim 1, wherein
the vibrated portion is fixedly supported to be vibratable via a supporting portion provided between the container portion and itself, and has a solidity higher than that of the supporting portion.

4. The container of claim 1, wherein
the vibrated portion comprises a contact surface outside the container portion, to which vibration is transmitted from outside, and the contact surface is provided with a high-polymer resin film.

5. The container of claim 1, wherein
the vibrated portion comprises a connected portion outside the container portion, to which vibration is transmitted from outside, and the connected portion is formed as a projection or a recess according to a recess or a projection of the external vibration generator to be connected, to fit each other by engaging.

6. The container of claim 1, wherein
the vibrated portion comprises a connected portion outside the container portion, to which vibration is transmitted from outside, and the connected portion is connected to the external vibration generator to be connected, by electromagnetic force.

7. The container of claim 1, wherein
the vibrated portion is integrally molded with the container portion.

8. The container of claim 1, wherein
the vibrated portion is joined to the container portion through the supporting portion, and the support portion is formed to have a flexible structure which supports the vibrated portion to be vibratable.

9. The container of claim 1, wherein
the vibrated portion comprises a rib projecting towards an outer circumference thereof, and the support portion comprises a reception groove which receives and covers the rib.

10. The container of claim 1, wherein
the vibrated portion comprises an inner surface portion, which defines an inner surface of the chamber.

11. The container of claim 1, wherein
the vibrated portion has a first intrinsic frequency of the vibration and the container or support portion has a second intrinsic frequency lower than the first intrinsic frequency.

12. The container of claim 1, wherein
the container portion is sealed with a lid.
